(19) United States
(12) Patent Application Publication
(10) Pub. No.: US 2007/0281925 A1
(43) Pub. Date: Dec. 6, 2007

(54) USE OF SERTINDOLE FOR THE PREVENTIVE TREATMENT OF SUICIDAL BEHAVIOUR

(75) Inventors: Mogens Sloth Nielsen, Bagsvaerd (DK); Mondher Toumi, Paris (FR); Anders Gersel Pedersen, Charlottenlund (DK)

Correspondence Address: LUNDBECK RESEARCH USA, INC.;ATTENTION: STEPHEN G. KALINCHAK, LEGAL, 215 COLLEGE ROAD, PARAMUS, NJ, 07652, US

(73) Assignee: H. Lundbeck A/S, Valby-Copenhagen (DK)

(21) Appl. No.: 11/756,100

(22) Filed: May 31, 2007

Related U.S. Application Data
(60) Provisional application No. 60/803,697, filed on Jun. 1, 2006.
(60) Provisional application No. 60/878,254, filed on Jan. 3, 2007.

(30) Foreign Application Priority Data
Jun. 1, 2006 (DK) PA 2006 00748
Dec. 28, 2006 (DK) PA 2006 01715

(51) Int. Cl. A61K 31/551; A61K 31/519; A61K 31/405

(52) U.S. Cl. 514/220; 514/419; 514/259.41

(57) ABSTRACT

The present invention relates to the use of sertindole in the preparation of a pharmaceutical composition for the preventive treatment of suicidal behaviour in human subjects suffering from a psychotic illness such as schizophrenia or schizoaffective psychosis.

This application claims the benefit of priority of U.S. Provisional Application No. 60/878,254, filed Jan. 3, 2007 and U.S. Provisional Application No. 60/803,697, filed Jun. 1, 2006; and claims the benefit of priority of Danish Application No. PA200601715, filed Dec. 28, 2006 and Danish Application No. PA200600748, filed Jun. 1, 2006, the contents of which are hereby incorporated by reference into the subject application.

### FIELD OF THE INVENTION

The invention relates to the use of sertindole for the preparation of a pharmaceutical composition for the preventive treatment of suicidal behaviour in a human subject who suffers from a psychotic illness such as schizophrenia or schizoaffective psychosis, a method for treating a suicidal behaviour in said human subjects, and a therapeutic package comprising sertindole and instructions for its use in treating a suicidal behavior

### BACKGROUND OF THE INVENTION

Sertindole, chemically named 5-chloro-1-(4-fluorophenyl)-3-(1-(2-(2-imidazolinon-1-yl)ethyl-4-piperidyl-1H-indole, is an antipsychotic drug with high affinity for serotonin 5-HT₂, dopamine D₂ and α₁-adrenergic receptors. Sanchez et al., Drug Dev Res. 1991; 22:239-250; Arnt J and Skarsfeldt T, Neuropsychopharmacol. 1998; 18(2):63-101. Sertindole is disclosed in U.S. Pat. No. Re. 34,299, and its antipsychotic activity is disclosed in U.S. Pat. No. 5,112,838. A method of manufacturing sertindole is disclosed in U.S. Pat. No. 6,335,463. Most research directed at the therapeutic effectiveness of sertindole has focused on its use in the treatment of schizophrenia. See, e.g., U.S. Pat. No. 5,112,838; Brown et al., Pharmacotherapy. 1993; 18(1):69-83; Samara, E. and Granneman, R., Clin. Pharmacol. & Therapeutics. 1996; 59(2):187; and Tamminga et al., International Clin. Psychopharmacol. 1997; 12(suppl. 1):S29-S35. Sertindole may also be effective in the treatment of other disorders such as: psychosis, including drug induced psychosis (U.S. Pat. No. 5,238,945); anxiety (U.S. Pat. No. 5,439,922); memory impairment (U.S. Pat. No. 5,444,073); substance dependency (U.S. Pat. No. 5,462,948); and depression, hypertension, and extrapyramidal side effects of other antipsychotic drugs (U.S. Pat. No. 5,703,087).

Sertindole reportedly has fewer adverse effects than antipsychotics such as haloperidol, fluphenazine and chlorpromazine. Brown et al., Pharmacotherapy. 1993; 18(1):69-83. This may be due to the limited binding of sertindole to histaminergic, muscarinic and α₂-adrenergic receptors, and to a regional electro-physiologic characteristic of the drug. Specifically, sertindole does not induce depolarization inactivation in A9 dopamine neurons (the nigrostriatal pathway), although the drug retains activity in A10 neurons (the mesolimbic and mesocortical pathways). The mesolimbic dopamine neurons are believed to mediate antipsychotic actions of neuroleptics, while the nigrostriatal neurons are believed to mediate motor side effects. Tamminga et al., International Clin. Psychopharmacol. 1997; 12(suppl. 1):S29-S35.

Patients with schizophrenia have, approximately, a 9 to 13% lifetime risk for successful suicide and, overall, a 50% lifetime risk for suicide attempts (Siris S G J Psychopharmacol 2001; 15:127-135; Nyman A K et al. Acta Psychiatr Scand 1986; 73:252-262). Patients who have made attempts to end their life in the past are particularly vulnerable to die from suicide at a later stage (Montross et al. Ann Clin Psychiatry 2005; 17:173-182). By comparison, the lifetime risk for suicide in the general population of the United States is approximately 1% (Caldwell C B et al. Suicide Life Threat Behav. 1992; 22:479-493).

Overall mortality rates in schizophrenia are approximately three to four times that of the general population (Meltzer H Y et al. Schizophr Bull 1999; 25:233-255) with suicide being one of the primary causes of death. A meta-analysis of increased mortality in patients with schizophrenia revealed that 59% of deaths were attributed to natural causes and 41% to unnatural causes (Brown S. Br J Psychiatry 1997; 171:502-508). Of those attributed to unnatural causes, 28% were the result of suicide.

Effects of Antipsychotic Medications on Suicide

Studies examining the effects of typical antipsychotics drugs on suicide and suicidal ideation have not identified changes in incidence with their use (Siris S G J Psychopharmacol 2001; 15:127-135; Caldwell C B et al. Suicide Life Threat Behav. 1992; 22:479-493; Axelsson R et al. Eur Arch Psychiatry Clin Neurosci 1992; 241:259-266; Winokur G et al. Am J Psychiatry 1975; 132:650-651; Johns C A et al. Ann N Y Acad Sci+ 1986; 487:294-300; Khan A et al. Int J Neuropsychopharmacol 2001; 4:113-118). It has been suggested that the use of typical antipsychotics may increase the risk of suicide, possibly due to of a combination of akathisia and depression (Caldwell C B et al. Suicide Life Threat Behav. 1992; 22:479-493; Shear M K et al. J Clin Psychopharmacol 1983; 3:235-236).

On the other hand, evidence exists that the atypical antipsychotic drug clozapine may reduce suicidal behaviour in human subjects with schizophrenia. (Meltzer H Y et al. Am J Psychiatry 1995; 152:183-190; Walker A M et al. Epidemiology 1997; 8:671-677; Reid W H et al. Psychiatr Serv 1998; 49:1029-1033; Munro J et al. Br J Psychiatry 1999; 175:576-580). The International Suicide Prevention Trial (InterSePT, Meltzer H Y Arch Gen Psychiatry 2003; 60:82-91) demonstrated that clozapine is superior to olanzapine in reducing the risk of suicide in both human subjects with schizophrenia and human subjects with schizoaffective disorder at high risk for suicide. Following the results of this study, the United States Food and Drug Administration (FDA) approved an expansion of the use of clozapine in December 2002 to include the treatment of recurrent suicidal behaviour in human subjects with schizophrenia and/or schizoaffective disorder who are at chronic risk.

The mortality rates and the effect of QTc interval of sertindole, risperidone and olanzapine were compared in a retrospective study presented as a poster at the International Congress on Schizophrenia Research in 1999 entitled: Suicide and Sudden Death During Treatment with Atypical Antipsychotics: A comparison of Sertindole, Olanzapine and Risperidone, by Moore N, Lagnaoui R, Toumi M and Bégaud B. The authors concluded i.a. that there were no obvious difference in the short-term (450 days) hazard functions between sertindole, olanzapine and risperidone, and that all-cause and cardiac death rates during clinical trial of these atypical antipsychotics were similar to those expected in this patient population.

WO 2004/082584 relates to the use of certain 5-HT2C receptor antagonists in the manufacture of medicaments for the treatment of mental disorders, in particular aspects of schizophrenia, cognitive impairment and suicidality, as well as to methods for determining the suitability of compounds for such a use. The patent application employs a mathematical model to make predictions of the effect of a long range of compounds on different mental disorders including suicidality, but presents no data in support hereof.

### DESCRIPTION OF THE INVENTION

The Sertindole Cohort Prospective (SCoP) study was designed as a multi-national, multi-centre, prospective, randomized, open-label, two parallel-group, active-controlled study in human subjects with schizophrenia. The objective of the study is to compare the safety of sertindole with the safety of another standard antipsychotic drug (in this case risperidone) under normal conditions of use. In addition, the study collects information on fatal or non-fatal suicide attempts. The study was initiated in 2002 and is still ongoing.

To ensure that the study population is representative of patients with schizophrenia, only a limited number of selection criteria are imposed. In particular, patients are required to fulfil the criteria specified in the SmPCs (Core Summary of Product Characteristics) for both sertindole and risperidone; they are not to have received either sertindole or risperidone as their treatment immediately before entering the study, and they require treatment with but a single anti-schizophrenia drug product. To ensure comparable populations in the two groups the patients are randomized to treatment via an interactive voice response system.

Data from the still ongoing Sertindole Cohort Prospective (SCoP) study have now shown, that the incidence of suicide attempts (both fatal and non-fatal) in schizophrenic patients on maintenance dose sertindole therapy is reduced significantly in comparison with human subjects treated with a standard antipsychotic drug, in this case risperidone.

This finding strongly indicates that sertindole has specific properties, which lead to a reduced frequency or incidence of fatal or non-fatal suicide attempts in schizophrenic patients.

The use of sertindole should thus lead to significantly fewer suicides in schizophrenic patients, but most likely also in individuals suffering from related psychotic illnesses such as schizoaffective psychosis. It has e.g. been shown that psychosis has a relationship to later suicidal activity for both schizophrenic and schizoaffective patients, and that psychosis may remain a risk factor for suicidal activity for schizoaffective patients, even when poor functioning is partialed out (Kaplan K J, Suicide Life Threat Behav. 1999 Spring; 29(1):10-24). Furthermore, it has been shown that chronic schizophrenia and schizoaffective psychosis are both very common diagnoses in persons who have committed or attempted to commit suicide (Flechtner K M et al., Nervenarzt. 1997 July; 68(7):569-73). Finally, the rate of attempted suicide in psychosis patients is very high (range 10% to 50%), (Caldwell C B et al., Schizophr Bull 1990; 16:571-89; Wilkinson G et al., Psychol Med 1984; 14:899-912) just as it is in schizophrenic individuals, in whom suicide is the leading cause of premature death (Radomsky E D, Am J Psychiatry 1999; 156:1590-5; Modestin J, Br J Psychiatry 1992; 160:867, 36; Tsuang M T, Arch Gen Psychiatry 1980; 37:979-83).

These findings indicate that the suicidal behaviour in schizophrenic patients is related to the suicidal behaviour of individuals suffering from other psychotic illnesses such as schizoaffective psychosis. Using sertindole in the treatment hereof should thus also lead to a lower suicide incidence rate in these other patient groups.

These findings are further corroborated by an exploratory analysis of clinical data obtained from a twelve-month open-label study (Tamminga C et al., “Identification of Potential Predictors of Sertindole Response in Patients with Schizophrenia”, to be published). In line with the emerging consensus criteria for response to antipsychotic treatment in schizophrenia (Andreasen et al., Am J Psychiatry 162:441-449, March 2005), the objective of this exploratory analysis is to identify potential predictors of response to sertindole in patients with schizophrenia.

The study assessed the safety and efficacy of sertindole doses (4-24 mg) administered once daily for up to one year in US patients with schizophrenia. Cox's regression analysis was applied to determine the effects of variables on time to sustained response (defined as achieving CGI-S<=3 and CGI-I<=2, both sustained for at least at least 8 weeks) in 358 patients.

125 patients achieved sustained response. Treatment with antipsychotic medication before first diagnosis of schizophrenia increased the response rate by 3-14% per year p.a. (p<0.01) compared with treatment-naive patients. Mildly or moderately ill patients were almost twice as likely (p<0.05) than more severely ill patients to respond to treatment with sertindole. Patients who had attempted suicide within the previous 5 years had a better treatment response than non-suicidal patients, whereas patients whose previous suicide attempt was at least 6 years earlier had a poorer response than non-suicidal patients (p<0.05). The analysis thus demonstrates the importance of initiating antipsychotic treatment before the patient becomes severely ill and also strongly suggests that efficient preventive treatment of suicidal behaviour relies on the treatment being initiated as soon as possible, once the suicidal behaviour has been diagnosed.

Accordingly, in one aspect the present invention relates to the use of sertindole for the preparation of a pharmaceutical composition for the preventive treatment of a suicidal behavior in a human subject who suffers from a psychotic illness.

In an embodiment the invention relates to the use of sertindole for the preparation of a pharmaceutical composition for the preventive treatment of a suicidal behavior in a human subject suffering from a psychotic illness who has attempted suicide at least once within the previous 5 years.

In a further aspect the present invention relates to a method of treating a suicidal behaviour in a human subject suffering from a psychotic illness, which comprises administering to said human subject an effective amount of a pharmaceutical composition comprising sertindole.

In one embodiment of the invention, the psychotic illness is schizophrenia. In another embodiment, the psychotic illness is schizoaffective psychosis. In yet another embodiment of the present invention, the suicidal behaviour can be treated or reduced using a combination of sertindole and one or more other antipsychotic drugs. In a further embodiment of the invention, the suicidal behaviour has resulted in one or more attempts at suicide within the previous 5 years. In a particular embodiment of the invention, the treatment may prevent human subjects altogether from committing or attempting to commit suicide.

In one embodiment, the pharmaceutical composition is administered orally as a once daily dosage form containing 4-24 mg of sertindole. In another embodiment the pharmaceutical composition is administered orally as a once daily dosage form containing 12-20 mg of sertindole

In a further embodiment of the present invention, the first initial dose of the pharmaceutical composition is about 4 mg sertindole/day. In another embodiment the first maintenance dose is reached by increasing the initial dose by about 4 mg/day. In an even further embodiment of the present invention, the next maintenance dose is reached by increasing the current dose by about 4 mg/day every 4-5 days until a maintenance dose of between about 12 mg/day and about 20 mg/day or the maximum dose (24 mg/day) is reached.

In another embodiment of the present invention, the established sertindole maintenance dose is changed by about 4 mg/day to establish a new maintenance dose. For example, a sertindole maintenance dose can be increased from 12 mg/day to 16 mg/day and, after one or more days, increased again by 4 mg/day to 20 mg/day. Thus, the dose of sertindole can be increased or decreased until an optimal dose (the new maintenance dose) or the maximum dose (24 mg/day) is reached.

According to a particular embodiment of the invention, the human subject has previously been treated with at least one other antipsychotic drug than sertindole, such as chlorpromazine, fluphenazine, haloperidol, thiothixene, trifluoperazine, perphenazine, and thioridazine, aripiprazole, risperidone, clozapine, olanzapine, quetiapine or ziprasidone.

In a further embodiment, the other antipsychotic drug is risperidone. In a still further embodiment, the other antipsychotic drug is olanzapine.

According to the present invention, the established maintenance dose can be about 4 mg/day to about 24 mg/day. Furthermore, according to the present invention, the established maintenance dose can be about 12 mg/day to about 20 mg/day.

Another aspect of the present invention is a method of marketing sertindole by marketing, advertising, or selling sertindole for the treatment or reduction of suicidal behaviour in a human subject in need thereof. The marketing may be directed to, for example, doctors (such as psychiatrists) treating human subjects suffering from a psychotic illness (such as schizophrenia or schizoaffective psychosis) who have a suicidal behaviour, and/or caretakers and relatives to such human subjects. The marketing step may comprise the step of including a statement in the labelling for a pharmaceutical product or composition containing sertindole that the product or composition can treat or reduce suicidal behaviour in a human subject suffering from a psychotic illness, such as schizophrenia or schizoaffective psychosis. Yet another aspect of the present invention is a therapeutic package, which comprises:
a) a dosage form of sertindole in one or more unit doses, such as tablets or capsules, in an amount such that periodic administration to the human subject is effective in treating his/her suicidal behaviour, such as 4-24 mg sertindole
b) a finished pharmaceutical container that contains said unit doses,
c) written matter such as labeling, directing the use of said package in the treatment of suicidal behaviour in human subjects suffering from a psychotic illness such as schizophrenia or schizoaffective psychosis.
d) an optional outside container or wrapper, such as a cardboard box

A still further aspect of the present invention is a method of dispensing sertindole to a human subject suffering from a psychotic illness, such as schizophrenia or schizoaffective psychosis, who displays a suicidal behaviour, which includes providing the human subject with a therapeutic package, which comprises:
a) a dosage form of sertindole in one or more unit doses, such as tablets or capsules, in an amount such that periodic administration to the human subject is effective in treating his/her suicidal behaviour, such as 4-24 mg sertindole
b) a finished pharmaceutical container that contains said unit doses,
c) written matter such as labeling, directing the use of said package in the treatment of suicidal behaviour in human subjects suffering from a psychotic illness such as schizophrenia or schizoaffective psychosis.
d) an optional outside container or wrapper, such as a cardboard box

In an embodiment of the invention the oral dosage form is in solid form such as capsules, tablets, lozenges, pills, gel capsules, granulates, powders and the like. In another embodiment the oral dosage form is in liquid form such as syrups, aqueous or non-aqueous solutions, suspensions and the like. In a further embodiment the dosage form is an injectable, sterile solution of sertindole meant for parental administration.

Terms and Definitions:

As used herein, “sertindole” comprises the free base or pharmaceutically acceptable salts of 5-chloro-1-(4-fluorophenyl)-3-(1-(2-(2-imidazolinon-1-yl)ethyl-4-piperidyl-1H-indole, in either crystalline or amorphous form, and solvates such as hydrates. Doses of sertindole as used herein always refer to the free base form unless otherwise specified. “A once daily dosage form containing 4-24 mg of sertindole” thus means “a once daily dosage form containing 4-24 mg of sertindole calculated as the free base”

As used herein, the term “pharmaceutically acceptable salts” includes acid addition salts or addition salts of free bases. Examples of acids which may be employed to form pharmaceutically acceptable acid addition salts include, but are not limited to, organic salts such as maleic, fumaric, benzoic, ascorbic, embonic, succinic, oxalic, bis methylene-salicylic, methanesulfonic, ethanedisulfonic, acetic, propionic, tartaric, salicylic, citric, glucomic, lactic, malic, mandelic, cinnamic, citraconic, aspartic, stearic, palmitic, itaconic, glycolic, p-amino-benzoic, glutamic, benzene sulfonic, theophylline acetic acids as well as the 8-halotheophyllines (for example 8-bromo-theophylline) salts; and inorganic salts such as hydrochloric, hydrobromic, sulfuric, sulfamic, phosphoric and nitric acid salts.

As used herein, a “pharmaceutical composition” comprises a solid or liquid mixture of sertindole with, optionally, pharmaceutically acceptable excipients, carriers and/or diluents such as water, monosaccharides, disaccharides, glycols, oils, or mixtures hereof, to which is optionally added colourings, aroma, preservatives, etc. provided that they are compatible with the active ingredient sertindole.

To prepare a pharmaceutical composition of the invention, an appropriate amount of sertindole is combined in an intimate admixture with a pharmaceutically acceptable carrier, which carrier can take a wide variety of forms depending on the form of preparation desired for administration. Such carriers are well known. These pharmaceutical compositions are desirably in unitary dosage form suitable for administration orally, nasally, rectally, percutaneously or by parenteral injection.

For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets, see e.g. “Remington's Pharmaceutical Sciences” by E. W. Martin, 18th Edition.

Because of their ease in administration, tablets and capsules represent the most advantageous oral dosage unit form, in which case solid pharmaceutical carriers are obviously employed.

It is especially advantageous to formulate the aforementioned pharmaceutical compositions in unit dosage form for ease of administration and uniformity of dosage. As used in the specification and claims, unit dosage form refers to physically discrete units suitable as unitary dosages, each unit containing a predetermined quantity of active ingredient(s) calculated to produce the desired therapeutic effect, in association with the required pharmaceutical carrier(s). Examples of such unit dosage forms are tablets (including scored or coated tablets), capsules, pills, powder packets, wafers, injectable solutions or suspensions, teaspoonfuls, tablespoonfuls and the like, and segregated multiples thereof.

As used herein, the term “psychotic illness” encompasses conditions, which are listed in DSM-IV (Diagnostic and Statistical Manual of Mental Disorders-4^{th} Ed., Am. Psych. Association).

As used herein, the term “Schizophrenia” comprises a disorder defined by DSM-IV and encompasses, but is not limited to, paranoid schizophrenia, disorganized schizophrenia, catatonic schizophrenia and undifferentiated schizophrenia. Schizophrenia is characterized by symptoms like delusions, hallucinations, disorganized speech, grossly disorganized or catatonic behaviour, negative symptoms. Positive symptoms of schizophrenia include, but are not limited to: delusions such as delusions of persecution, reference, thought withdrawal and thought insertion; hallucinations such as auditory, visual, olfactory, gustatory and tactile hallucinations; thought disorder; and bizarre behaviour. Negative, or deficit, symptoms of schizophrenia include, but are not limited to, blunted affect, poverty of speech, anhedonia and asociality.

As used herein, the term “Schizoaffective psychosis” means a disorder defined by DSM-IV characterized by the presence of symptoms of schizophrenia like delusions, hallucinations, disorganized speech, grossly disorganized or catatonic behaviour, negative symptoms together with mood symptoms like depression or mania.

As used herein, the term “antipsychotic drug” comprises a medication used to treat schizophrenia and psychotic symptoms (such as hallucinations, delusions and other positive symptoms as defined in DSM-IV) in other psychiatric illnesses, and further comprises both typical and atypical antipsychotic drugs.

As used herein, the term “typical antipsychotic drug” encompasses, but is not limited to chlorpromazine (Thorazine), fluphenazine (Prolixin), haloperidol (Haldol), thiothixene (Navane), trifluoperazine (Stelazine), perphenazine (Trilafon), and thioridazine (Mellaril).

As used herein, the term “atypical antipsychotic drug” encompasses, but is not limited to aripiprazole (Abilify), risperidone (Risperdal), clozapine (Clozaril), olanzapine (Zyprexa), quetiapine (Seroquel), sertindole (Serdolect) and ziprasidone (Geodon).

As used herein, the term “maintenance dose” means a constant daily dose (i.e., the same dose amount is administered each day, typically between 4-24 mg/day) of sertindole sufficient to maintain the desired activity of sertindole in a human subject suffering from a disease or condition. The maintenance dose is an optimal dose that is administered to a human subject on a continuing, daily basis. For example, in a human subject under treatment for schizophrenia, the optimal dose is determined by observing an improvement or stabilization in the positive and/or negative symptoms of schizophrenia, in the absence of dose-limiting side effects such as, for example, rhinitis, nasal congestion, abnormal ejaculation, postural hypotension, weight gain, peripheral edema, dyspnea, or QTc interval prolongation. Thus, a maintenance dose is established when an optimal dose is determined and the optimal dose is administered to the human subject on a daily basis. A maintenance dose is also established when an optimal dose is determined and a physician prescribes daily administration of the optimal dose to the human subject.

As used herein, the term “combination” refers to the treatment of a human subject with sertindole and one or more other antipsychotic drugs, preferably an atypical antipsychotic drug such as aripiprazole, risperidone, clozapine, olanzapine, quetiapine or ziprasidone.

As used herein, the term “suicidal behaviour” comprises performing actions that put him/herself at risk of death. Suicidal behaviour may include acts of self-harm with a fatal (suicide) or a nonfatal (attempted suicide) outcome.

As used herein, the phrases “preventive treatment of suicidal behavior” and “treating suicidal behaviour” mean administering a pharmaceutical composition comprising sertindole to a human subject who has a suicidal behavior with the result that said suicidal behavior is reduced, thereby leading to a lower frequency of suicide attempts or even to a complete prevention of the human subject committing or attempting to commit suicide.

As used herein, the term “finished pharmaceutical container” comprises devices suitable for the storage of 1) solid pharmaceutical dosage forms such as capsules, tablets, lozenges, pills, gel capsules, granulates, powders and the like and 2) liquid pharmaceutical dosage forms such as syrups, aqueous or non-aqueous solutions, suspensions and the like, including sterile injectable solutions. The term encompasses, but is not limited to containers, blister packs, pill boxes, bottles, jars, safety packings, tablet dispensers, ampoules, foil wrappings, pill or medicine organizers, dispensers and managers, pill fobs and totes.

As used herein, the term “unit dose” comprises solid or liquid pharmaceutical dosage forms of sertindole such as capsules, tablets, lozenges, pills, gel capsules, granulates, powders, syrups, aqueous or non-aqueous solutions, suspensions and sterile injectable solutions; preferably tablets, each containing 4 mg, 12 mg, 16 mg or 20 mg sertindole

As used herein, the term “therapeutic package” comprises:
a) a dosage form of sertindole in one or more unit doses in an amount such that periodic administration to the human subject is effective in treating his/her suicidal behaviour
b) a finished pharmaceutical container that contains said unit doses,
c) written matter such as labeling, directing the use of said package in the treatment of suicidal behaviour in human subjects suffering from a psychotic illness such as schizophrenia or schizoaffective psychosis.
d) an optional outside container or wrapper, such as a cardboard box

As used herein, the term “written matter” encompasses, but is not limited to, package inserts, labels, patient brochures, patient leaflets, user manuals and videotapes

As used herein, the term “advertising” refers to notifying, informing, and/or apprising one or more individuals of information (e.g., the efficacy of a pharmaceutical product for treating or reducing an indication), such as by mass media, including, but not limited to, newspaper, magazine, and internet advertisements, television commercials, and billboard signs. The term “advertising” as used herein also includes including a statement that the pharmaceutical product can treat or reduce the indication in the labelling for the pharmaceutical product.

As used herein, the term “marketing” refers to the act or process of selling a product, including, but not limited to, any offer for sale or sale of a product.

Experimental Procedures

Dose and Administration

Sertindole (manufactured by H. Lundbeck A/S, Copenhagen-Valby, Denmark) (Serdolect®, Zerdol®) is supplied as 4 mg, 12 mg, 16 mg and 20 mg film-coated tablets. Sertindole is typically administered orally once daily with or without food. Generally, patients should be started on sertindole at about 4 mg/day, and the dose should be increased by about 4 mg/day after every 4-5 days until the optimal daily dose of about 12 mg/day to about 20 mg/day is reached. The optimal dose is continued and is the maintenance dose. The maximum daily dose of sertindole is about 24 mg/day (cf. Core Summary of Product Characteristics, Sep. 7, 2005, “SmPC”). As well known to one of ordinary skill in the art, the starting dose and subsequent doses of sertindole can vary depending on, for example, disease or condition severity; and the age, weight, physical condition and responsiveness of the patient to be treated.

Sertindole Cohort Prospective (SCoP) study design

The Sertindole Cohort Prospective (SCoP) study is a multinational, multi-centre, prospective, randomized, open-label, two parallel-group, active-controlled study in patients with schizophrenia. The objective of the study is to compare the safety of sertindole with the safety of another standard antipsychotic drug (in this case risperidone) under normal conditions of use. In addition, the study collects information on fatal or non-fatal suicide attempts. The study was initiated in 2002 and is still ongoing.

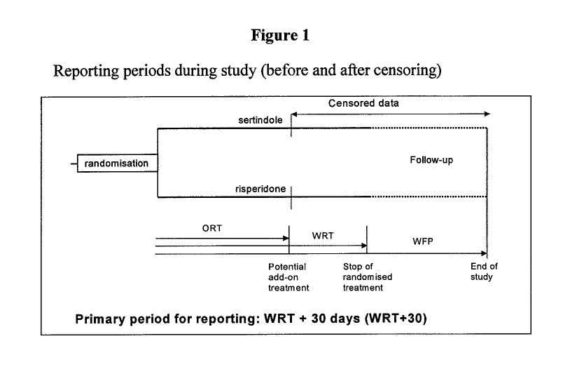

The data in the study are reported for different periods. The Only-Randomized-Treatment-Period (ORT) covers the time the patient is receiving antipsychotic monotherapy with the randomized trial medication. However, patients can also be treated in combination with another antipsychotic if this is clinically necessary. When the patient receives an additional antipsychotic, the patient is censored and the data are reported for the Whole-Randomized-Treatment period (WRT). The primary period for reporting is the so-called Whole-Randomized-Treatment+30 period (WRT+30), which covers the time during which the patient receives randomized treatment plus 30 days after treatment discontinuation. Finally, the period during which the patient is followed even after the treatment has been stopped is called the Whole-Follow-up-Period (WFP). The reporting periods in the trial are presented in FIG. 1 below.

To ensure that the study population is representative of patients with schizophrenia, only a limited number of selection criteria are imposed. In particular, patients are required to fulfill the criteria specified in the SmPCs for both sertindole and risperidone; they are not to have received either sertindole or risperidone as their treatment immediately before entering the study, and they require treatment with but a single antipsychotic drug. To ensure comparable populations in the two groups the patients are randomized to treatment via an interactive voice response system.

Throughout the treatment period patients are dosed according to the treatment recommendations in their respective SmPCs.

Data Analysis

An analysis of the two most robust measures of risk of suicide, i.e. completed suicide and attempted suicide, has been performed using data from the ongoing SCoP study up until 11 Jan. 2006 and 11 Jan. 2007, respectively.

Regarding attempted suicide, the analysis comprised only those events for which the investigators confirmed that the patients actually tried to commit suicide. Events such as intentional overdose and intentional self-injury were clarified by the investigators and were not included in the analysis unless the investigators confirmed that they were real suicide attempts. The included events were coded as ‘suicide attempt’ in the database.

The above definition of attempted suicide has the advantage of making it possible combine this event with completed suicide and to analyze the time until either event occurs. This can be done since all patients are randomised to start new treatment with either sertindole or risperidone.

All events occurring in the Whole Randomised Treatment (WRT) period plus 30 days were considered for analysis, up until the data cut-off. The WRT period is the period from start to stop of randomised treatment. The null hypothesis of equality of hazards between treatment groups was tested using the log-rank test.

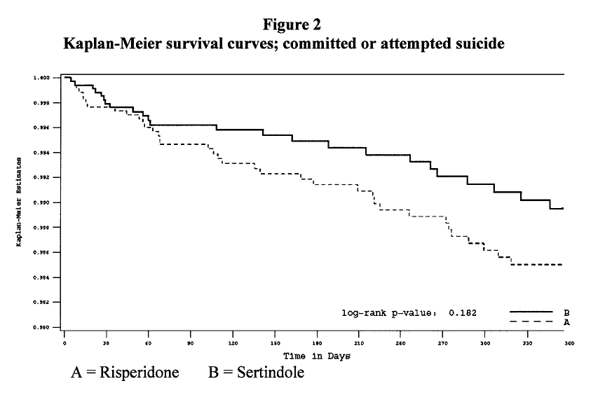

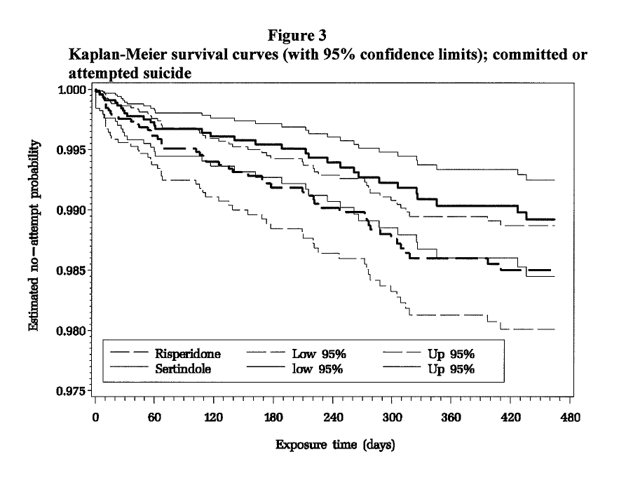

The observed number of events subject to analysis is shown in Table 1 and Table 3. The results from the time to event analysis are shown in Table 2 and Table 4, and the corresponding Kaplan-Meier survival curves are shown in FIG. 2 and FIG. 3.

TABLE 1
Patients committing suicide or attempting suicide as of Nov. 1, 2006

| Event | SERT | RISP* |
|---|---|---|
| Patient years exposure (PYE) | 3168 | 3473 |
| Committed or attempted suicide | 27 (0.85) | 40 (1.15) |
| (n (rate/100 PYE)) | | |

SERT = sertindole,
RISP = risperidone
*One patient attempted suicide and then completed suicide at a later time point

TABLE 2
Log-rank test of equality of hazards between the two treatment groups

| | Hazard ratio* | | | |
|---|---|---|---|---|
| | | Lower | Upper | |
| Event | Ratio | 95% CI | 95% CI | p-value |
| Committed or attempted suicide | 0.72 | 0.44 | 1.17 | 0.182 |

*SERT/RISP

The estimated hazard ratio was approximately 0.7 or, equivalently, the event rate was nearly 1.5 times higher in the risperidone group than in the sertindole group.

An update of the results for suicide attempt (fatal or non-fatal) is presented below using data collected up until the data cut-off at 11.1 2007.

TABLE 3
Patients committing suicide or attempting suicide as of Nov. 1, 2007

| Event | SERT | RISP* |
|---|---|---|
| Patient years exposure (PYE) | 5344 | 5549 |
| Committed or attempted suicide | 33 (0.62) | 51 (0.92) |
| (n (rate/100 PYE)) | | |

SERT = sertindole,
RISP = risperidone
*One patient attempted suicide and then completed suicide at a later time point

TABLE 4
Log-rank test of equality of hazards between the two treatment groups

| | Hazard ratio* | | | |
|---|---|---|---|---|
| | | Lower | Upper | |
| Event | Ratio | 95% CI | 95% CI | p-value |
| Committed or attempted suicide | 0.69 | 0.45 | 1.07 | 0.0976 |

*SERT/RISP

Again, the estimated hazard ratio was approximately 0.7 or, equivalently, the event rate was nearly 1.5 times higher in the risperidone group than in the sertindole group.

Further results from analysis of time to suicide attempt (fatal or non-fatal), when using Cox's proportional hazards model on the data collected up until the data cut-off at 11.1 2007, are presented in Table 5. In this model, adjustment of the treatment effect for baseline imbalances has been performed for the following variables: age, gender, total duration of schizophrenia (<5 years, 5-10 years, >10 years), and time since last suicide (never attempted suicide, last attempt <1 year ago, last attempt 1-5 years ago, last attempt more than 5 years ago), which may all be important prognostic factors for suicide attempt.

TABLE 5
Patients committing suicide or attempting suicide as of Nov. 1, 2007;
Cox proportional hazards model adjusting for each patient's age,
gender, history of suicide attempt, and duration of schizophrenia

| | | 95% | | |
|---|---|---|---|---|
| | | Hazard Ratio | | |
| | Hazard | Confidence | | |
| Parameter | Ratio | Limits | | p-value |
| SERT as compared to RISP | 0.614 | 0.391 | 0.965 | 0.0344 |
| Age | 0.994 | 0.971 | 1.017 | 0.6017 |
| Female as compared to Male | 0.822 | 0.523 | 1.293 | 0.3971 |
| Time since last suicide attempt | 16.694 | 9.565 | 29.137 | <.0001 |
| Attempt <1 yr as compared to never | | | | |
| attempted suicide | | | | |
| Time since last suicide attempt | 7.672 | 4.271 | 13.781 | <.0001 |
| Attempt 1-5 yrs as compared to | | | | |
| never attempted suicide | | | | |
| Time since last suicide attempt | 3.218 | 1.420 | 7.293 | 0.0051 |
| Attempt >5 yrs as compared to never | | | | |
| attempted suicide | | | | |
| Total duration of schizophrenia 5-10 | 1.140 | 0.667 | 1.950 | 0.6312 |
| years as compared to <5 years | | | | |
| Total duration of schizophrenia | 0.785 | 0.412 | 1.493 | 0.4603 |
| >10 years as compared to <5 years | | | | |

In this model the estimated hazard ratio of the risk of suicide attempt in the sertindole group as compared to that in the risperidone group was approximately 0.6, which was statistically significant (p=0.0344). The event rate was approximately 1.7 times higher in the risperidone group than in the sertindole group.

1. (canceled)

2. The method according to claim 17, wherein the treatment prevents human subjects from committing or attempting to commit suicide.

3. The method according to claim 17, wherein the treatment reduces the risk of a human subject committing or attempting to commit suicide.

4. The method according to claim 17, wherein the human subject has attempted suicide at least once within the previous 5 years.

5. The method according to claim 4, wherein the human subject has previously been treated with at least one other antipsychotic drug than sertindole.

6. The method according to claim 5, wherein the other antipsychotic drug is risperidone.

7. The method according to claim 5, wherein the other antipsychotic drug is olanzapine.

8. The method according to claim 17, wherein the suicidal behavior is treated or reduced using a combination of sertindole and another antipsychotic drug.

9. The method according to claim 8, wherein the pharmaceutical composition is administered as a once daily dosage form.

10. The method according to claim 9, wherein the pharmaceutical composition is a unit oral dosage form.

11. The method according to claim 10, wherein said pharmaceutical composition is associated with instructions stating that sertindole is indicated for treating a suicidal behavior in human subjects suffering from a psychotic illness.

12. A therapeutic package comprising:
(a) a dosage form of sertindole in one or more unit doses, wherein each unit dose comprises an amount of sertindole effective in treating the patient's suicidal behavior.
(b) a finished pharmaceutical container containing the unit doses of sertindole;
(c) written matter associated with the therapeutic package stating that the dosage form can be administered to treat a suicidal behavior in a human subject suffering from a psychotic illness.
(d) an optional outside container or wrapper.

13. (canceled)

14. The therapeutic package according to claim 12, wherein said written matter states that the dosage form can be administered for the preventive treatment of suicidal behavior in a human subject who suffers from schizophrenia.

15. The therapeutic package according to claim 12, wherein said written matter states that the dosage form can be administered for the preventive treatment of suicidal behavior in a human subject who suffers from schizoaffective psychosis.

16. A method for marketing sertindole comprising the step of marketing sertindole for the treatment of suicidal behavior in human subjects suffering from a psychotic illness.

17. A method of treating a suicidal behavior in a human subject suffering from a psychotic illness, which comprises administering to said human subject an effective amount of a pharmaceutical composition comprising sertindole.

18. A method of dispensing sertindole to a human subject suffering from a psychotic illness who displays a suicidal behavior, which comprises providing the human subject with a therapeutic package according to claim 12.

19. The method according to claim 16, wherein the psychotic illness is schizophrenia.

20. The method according to claim 16, wherein the psychotic illness is schizoaffective psychosis.

21. The method according to claim 17, wherein the psychotic illness is schizophrenia.

22. The method according to claim 17, wherein the psychotic illness is schizoaffective psychosis.

23. The method according to claim 10, wherein the unit oral dosage form is between about 4 mg to about 24 mg of sertindole.

24. The method according to claim 23, wherein said unit oral dosage form is a maintenance dose.

25. A therapeutic package according to claim 12, wherein each unit dose comprises an amount about 4 mg to about 24 mg of sertindole.

26. A therapeutic package according to claim 12, wherein the written matter is a label.

27. The method according to claim 18, wherein the psychotic illness is schizophrenia.

28. The method according to claim 18, wherein the psychotic illness is schizoaffective psychosis.
